(19) United States
(12) Patent Application Publication
(10) Pub. No.: US 2026/0138131 A1
(43) Pub. Date: May 21, 2026

(54) ANTIBIOTIC SUSCEPTIBILITY TESTING REAGENT DISC STRUCTURE AND METHOD THEREOF

(71) Applicant: BIOGUARD CORPORATION, New Taipei City (TW)

(72) Inventor: YU-CHEN CHOU, New Taipei City (TW)

(21) Appl. No.: 19/031,148

(22) Filed: Jan. 17, 2025

(30) Foreign Application Priority Data
Nov. 15, 2024 (TW) 113144086

(51) Int. Cl. B01L 3/00

(52) U.S. Cl. CPC B01L 3/50273; B01L 2200/0684; B01L 2200/16; B01L 2300/0803; B01L 2400/0409

(57) ABSTRACT

An antibiotic susceptibility testing reagent disc structure includes a test carrier, at least two supporting and positioning portions, a central injection portion, a plurality of micro flow channels, a plurality of reaction receptacles, a plurality of preloaded reagents for AST, a plurality of overflow channels, a plurality of overflow troughs, a plurality of narrowing portions, and a plurality of vent holes. Before packaging, the reaction receptacles of the test carrier are preloaded with freeze-dried preloaded reagents. A user can directly position the test carrier in the testing device after injecting the specimen into the central injection portion. Upon activation, the specimen is acted upon by the centrifugal force to evenly flow through the micro flow channels into the reaction receptacles. For an amount of specimen contained in the reaction receptacles is larger than the volume thereof, it flows along the overflow channels into the overflow troughs.

### BACKGROUND OF THE INVENTION

### (a) Technical Field of the Invention

The present invention provides an antibiotic susceptibility testing reagent disc structure, and a method thereof, which simplify pre-test steps, enable simultaneous performance of multiple antibiotic susceptibility tests, evenly distribute specimen in an appropriate amount, provide a space for specimen to react independently and a vent hole so as to achieve simple operation, uniform concentration, sufficient bacterial volume, and avoiding cross-contamination of specimens and reducing the inability to interpret results due to human operation techniques.

### (b) Description of the Prior Art

Antibiotic Susceptibility Testing (AST), also known as antibiotic sensitivity testing, is a test designed to assess the sensitivity of bacteria, fungi or other microorganisms to antibiotic drugs (such as antibiotics) in order to select the most effective treatment options.

The traditional AST process is to first culture bacteria to be diluted to a usable concentration, and a culture dish is prepared. The diluted bacterial liquid is dropped into the culture dish for coating, and an antibiotic of a known concentration is then added into the culture dish for growing and reacting at an appropriate temperature for a period of time. Finally, the size of the inhibition zone is measured to record and judge with the naked eye whether the antibiotic is an effective therapeutic drug for the bacteria.

Currently, there are an AST test device and a disc thereof available on the market, claiming to be able to complete a test quickly. The operation steps are to collect a specimen first, and then completing dilution thereof. Afterwards, an indicator is dropped into the bacterial liquid for mixing, and then injected into the disc to be eventually placed into the device for reaction, and judgement can be made according to the color of the reagent after the reaction is completed.

The above AST test device and the disc thereof have the following problems and deficiencies when used and need to be further improved:

Firstly, although the operation is faster than the traditional processes, it is easy to have problems with personal operation techniques, resulting in nonuniform indicator concentration and uneven coloring, making the result interpretation problematic. In fact, for operators, there are still four or five steps, and the numerous steps will easily lead to contamination or contamination.

Secondly, the volume of each reaction receptacle on the disc is less than 20µl, which causes the pathogenic bacteria to be unable to grow normally due to the small amount of bacteria during the reaction. On the other hand, the strong competition of antibiotics causes the bacteria to be unable to grow normally, resulting in the test results of the disc cannot be interpreted.

Thirdly, the reaction receptacles on the disc are interconnected, which can easily cause cross contamination. Moreover, it is operated through diffusing naturally from the first reaction receptacle to the last reaction receptacle, so that it is easy to cause nonuniform bacterial liquid concentration and relatively significant difference of bacterial amount among the reaction receptacles. The indicator and specimen between the reaction receptacles will affect each other, making the result uninterpretable.

Fourthly, the disc is not provided with a vent hole. As the bacteria will produce gas during the reaction process, and the space inside the disc is closed. Once the gas is not discharged in time, due to that the more bacteria there are, the more gas there will be, a membrane on the upper layer of the disc will be compressed to break open the disc, causing leakage to result in contamination.

### SUMMARY OF THE INVENTION

Thus, in view of the above problems, the present inventor has collected relevant information, evaluated and considered various aspects, and based on many years of experience in this industry, creates an antibiotic susceptibility testing reagent disc structure, and a method thereof, which simplify pre-test steps, enable simultaneous performance of multiple antibiotic susceptibility tests, evenly distribute specimen in an appropriate amount, provide a space for specimen to react independently and a vent hole so as to achieve simple operation, uniform concentration, sufficient bacterial volume, and avoiding cross-contamination of specimens and reducing the inability to interpret results due to human operation techniques.

The main purpose of the present invention is to make use of the independence realized through micro flow channels being arranged in a radiating form at intervals, allowing disposition and setup in a test device to be carried out immediately after specimen is introduced through one single central injection portion, making operation steps extremely simplified and evenly distributing the specimen quantity.

Another main purpose of the present invention is that in each of the reaction receptacles of 20µl-80µl, an equal amount of preloaded reagent is loaded, so as to save the step of manually introducing reagent, ensuring identical dosage and concentration of reagent in each of the reaction receptacles, and consistency of bacterial liquid amount in each of the reaction receptacles, making the result of AST experiments highly complete and accurate.

A further main purpose of the present invention is to make use of an arrangement of overflow channels and overflow troughs to allow an amount of specimen exceeding the volume of the reaction receptacles to flow toward the overflow troughs, so as to effectively control the bacteria amount to heighten the accuracy of experiment and prevent cross-contamination resulting from back-flowing of the bacterial liquid.

Yet a further main purpose of the present invention is to make use of an arrangement of vent holes to allow gas generated during the reaction process to discharge out, in order to prevent the test carrier from being damaged through collapse resulting from an excessive amount of gas and thus indirectly avoiding contamination caused by liquid leakage.

To achieve the above purposes, a structure of the present invention comprises a test carrier, at least two supporting and positioning portions, a central injection portion for introducing a specimen, a plurality of micro flow channels arranged in a radiating form and independent of each other, a plurality of reaction receptacles, a plurality of preloaded reagents for AST, a plurality of overflow channels, a plurality of overflow troughs, a plurality of narrowing portions, and a plurality of vent holes. The central injection portion is arranged on the test carrier. The supporting and positioning portions are formed in the test carrier. The micro flow channels are arranged in a radiating form at intervals beside the central injection portion and each in communication therewith. The reaction receptacles are respectively arranged on the micro flow channels. The preloaded reagents are loaded in the reaction receptacles. The overflow channels are respectively formed at one side of the reaction receptacles that is opposite to the micro flow channels. The overflow troughs are respectively formed at one side of the overflow channels and in communication therewith. The narrowing portions are respectively formed on the overflow channels. The vent holes are respectively arranged at one side of the overflow troughs and in communication therewith.

When a user uses the present invention to conduct antibiotic susceptibility testing, as the reaction receptacles of the test carrier are pre-loaded with the freeze-dried preloaded reagents before packaging, the user only needs to introduce the specimen through the central injection portion before positioning the test carrier into a testing device for activation. Further, during injection, as the supporting and positioning portions limit the insertion depth of the injection tool, damage to the sealing membrane can be avoided, while ensure the complete injection of the specimen. The pre-test steps are simple, and problems resulting from human operation techniques are basically eliminated. During rotation of the test carrier, the specimen is acted upon by the centrifugal force so as to flow through the plurality of micro flow channels that are arranged in a radiating form at intervals into each of the reaction receptacles that has a volume of 20µl-80µl. When the specimen completely flows out of the central injection portion, a portion of an individual amount of specimen in each of the reaction receptacles that is larger than the volume thereof flows through the overflow channel into the overflow trough corresponding thereto. Further, the arrangement of the narrowing portions reduces the maximum flow rate flowing into the overflow troughs. And then, the specimen reacts with the preloaded reagent in each of the reaction receptacles. The gas generated during the reaction is discharged out of the test carrier through the vent hole. As such, the specimen can be evenly distributed to each of the micro flow channels and the amount of specimen in each of the reaction receptacles can be stably controlled, together with the reaction receptacles being loaded in advance preloaded reagents that are freeze-dried, all the condition being make easy to handle, helping enhance completeness and accuracy of experiments, and the arrangement of independent channel and vent hole helps eliminate the potential risk of cross-contamination.

By means of the above technology, the problems existing in the prior art AST test device and a disc thereof that the pre-test steps are complicated, it is easily involved manual operation problems, the bacterial concentration is nonuniform, the bacterial volume is insufficient, removal of excessive gas is impossible, test accuracy is insufficient, and cross-contamination is easy can be overcome to achieve the practical progress mentioned above.

### BRIEF DESCRIPTION OF THE DRAWINGS

FIG. 1 is a perspective view showing a first preferred embodiment of the present invention.

FIG. 2 is a flow chart showing steps according to the first preferred embodiment of the present invention.

FIG. 3 is a schematic view showing reagent pre-loading according to the first preferred embodiment of the present invention.

FIG. 4 is a schematic view showing specimen injection according to the first preferred embodiment of the present invention.

FIG. 5 is a schematic view showing specimen splitting according to the first preferred embodiment of the present invention.

FIG. 6 is a schematic view showing specimen overflowing according to the first preferred embodiment of the present invention.

FIG. 7 is a schematic view showing reaction process according to the first embodiment of the present invention.

FIG. 8 is an exploded view showing a second preferred embodiment of the present invention.

FIG. 9 is an exploded view showing a third preferred embodiment of the present invention.

FIG. 10 is a perspective view, partly sectioned, showing a fourth preferred embodiment of the present invention.

FIG. 11 is a perspective view, in a see-through form, showing a fifth preferred embodiment of the present invention.

FIG. 12 is a perspective view, partly sectioned, showing the fifth preferred embodiment of the present invention from a different angle.

FIG. 13 is a plan view showing a sixth preferred embodiment of the present invention.

### DETAILED DESCRIPTION OF THE PREFERRED EMBODIMENTS

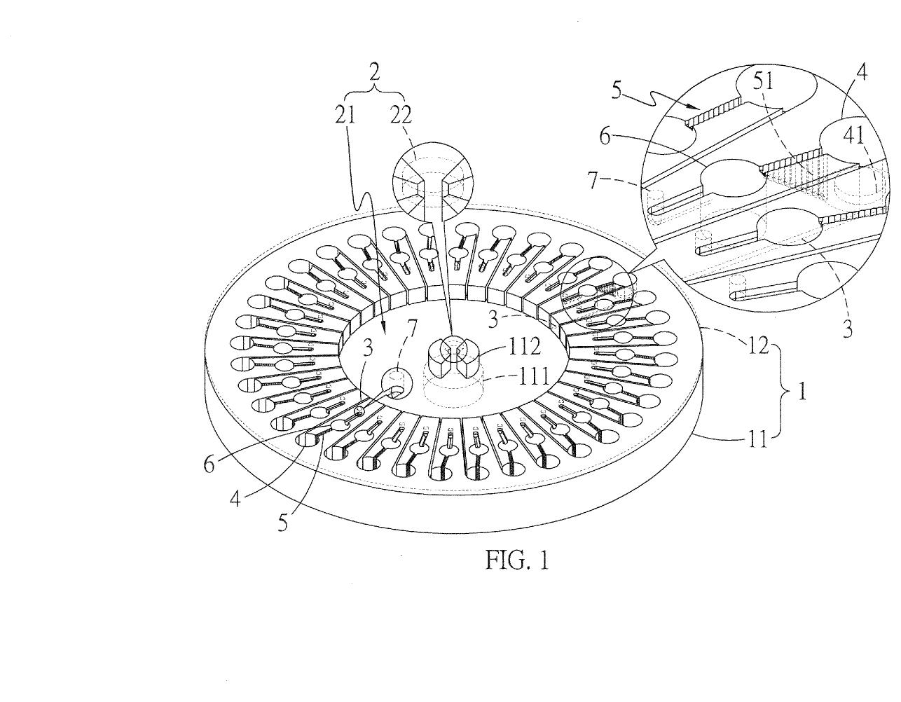

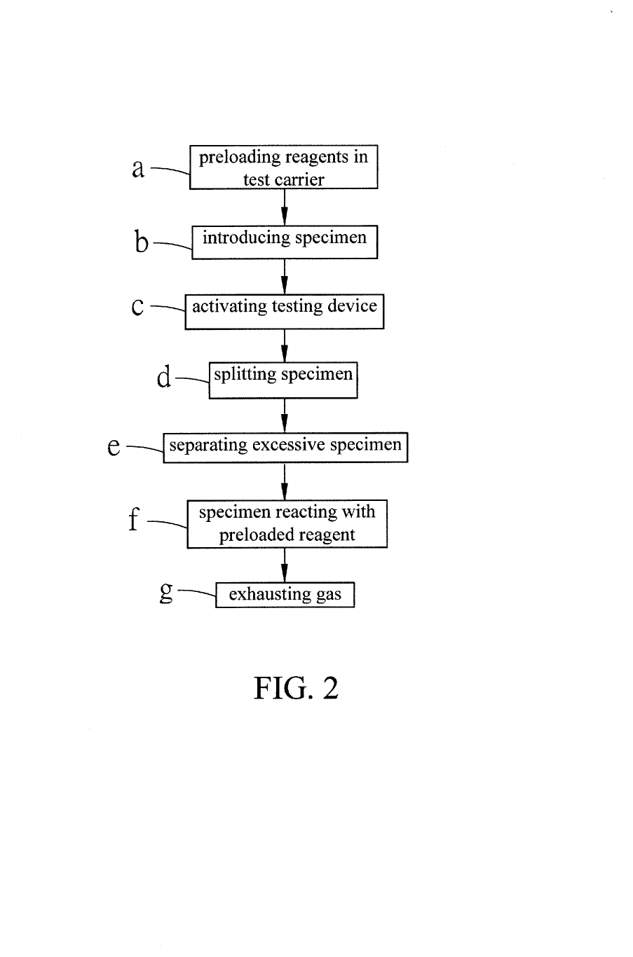

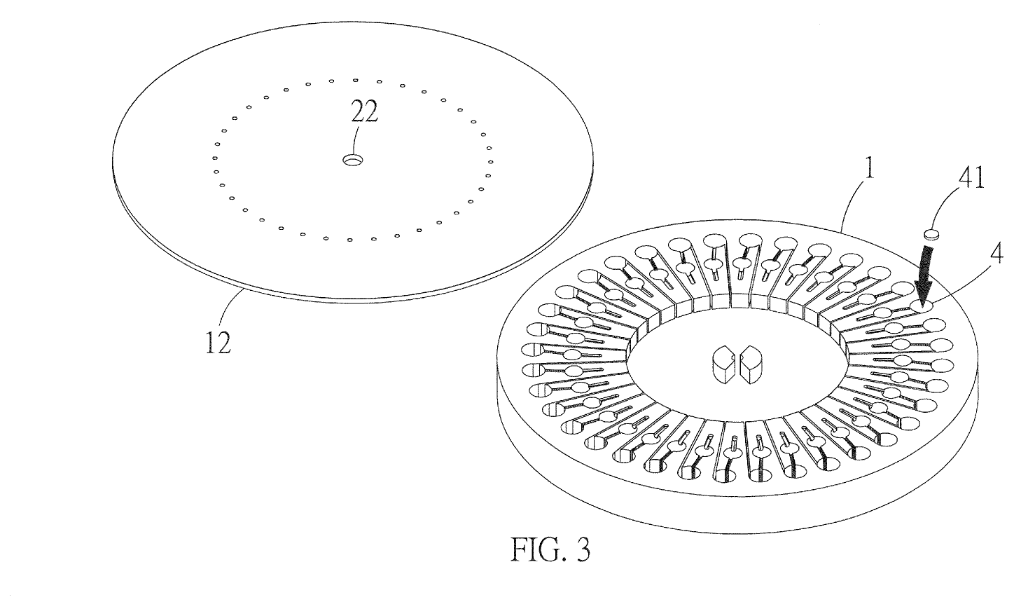

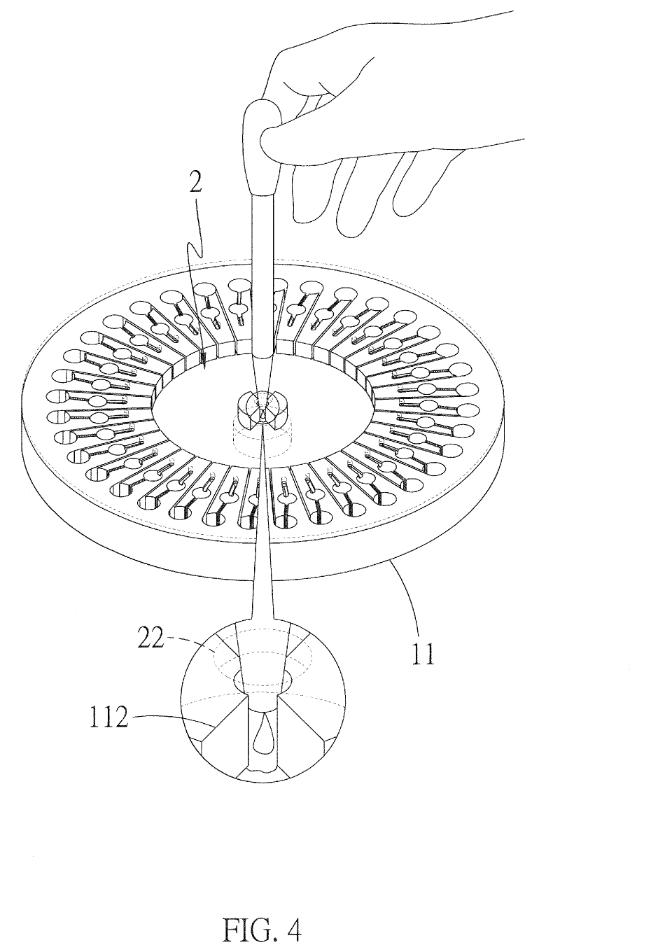

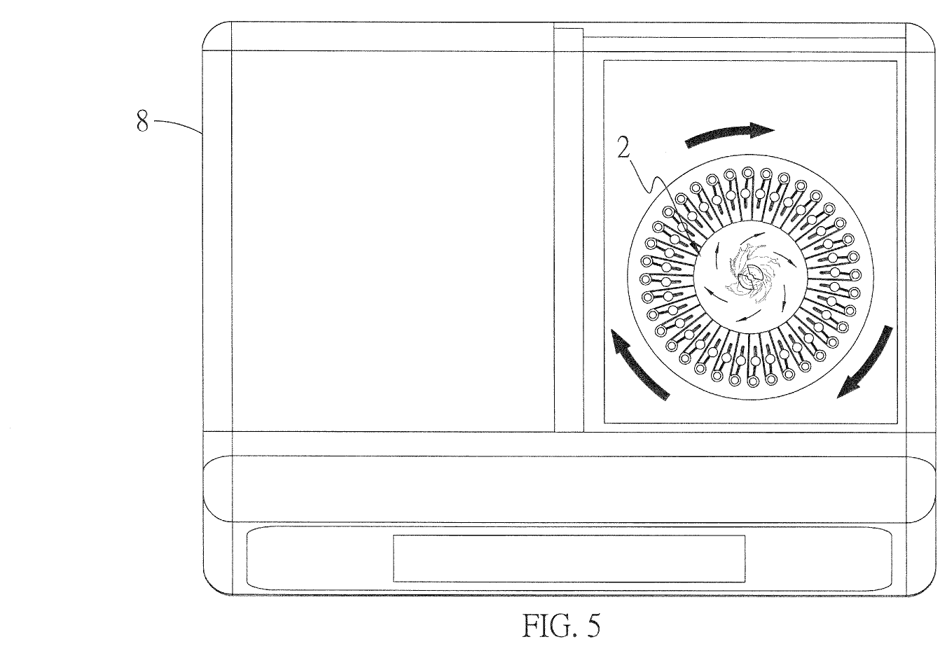

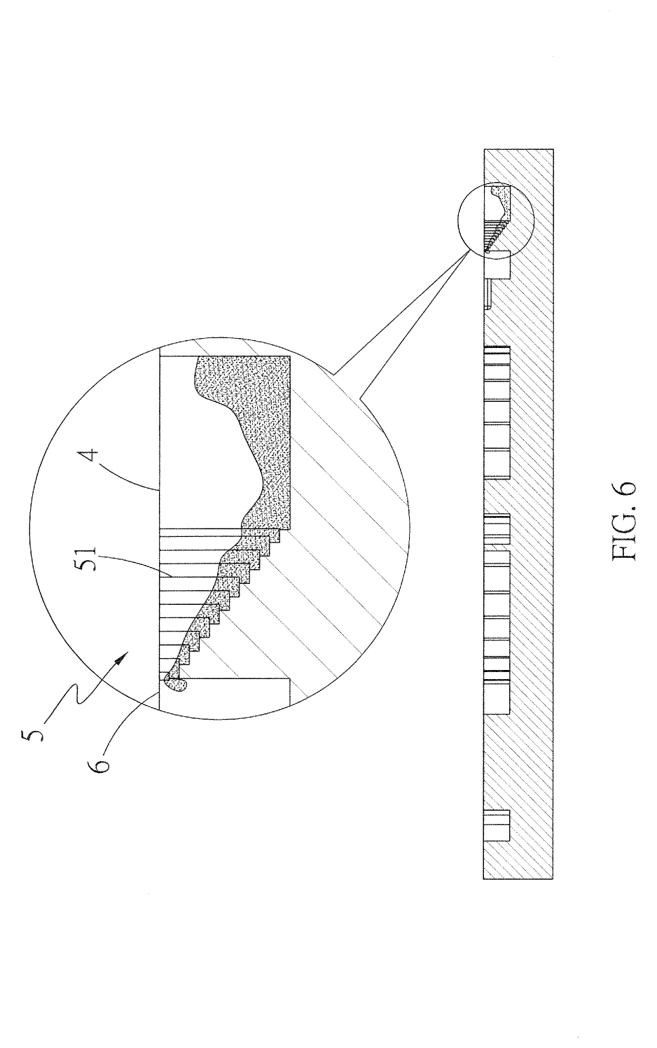

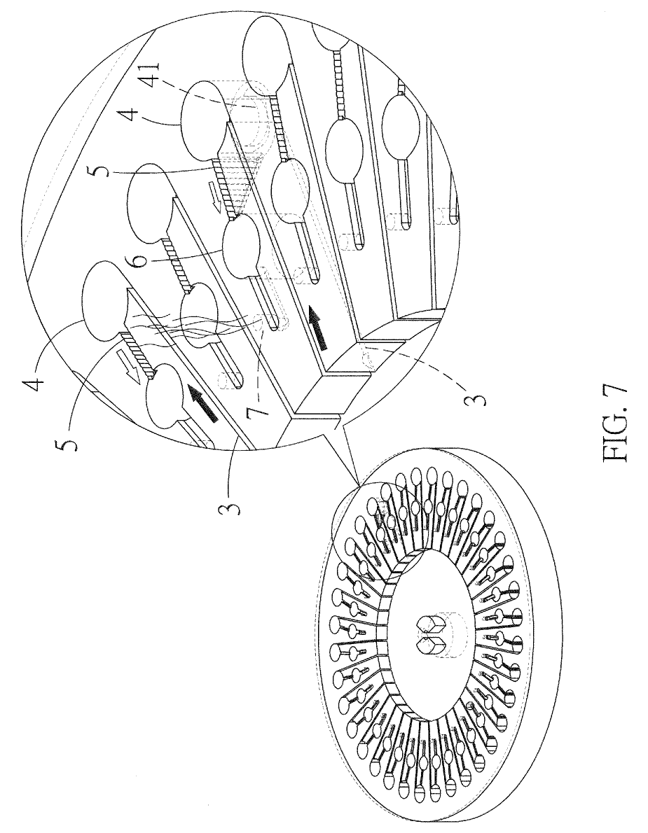

Referring to FIGS. 1-7, which are a perspective view to a schematic view showing reacting of a first preferred embodiment of the present invention, the present invention comprises: a test carrier 1; at least two supporting and positioning portions 112, which are formed in the test carrier 1 to support the test carrier 1; a central injection portion 2, which is arranged between the supporting and positioning portions 112 for introduction of a specimen and limiting an insertion depth of an injection tool by means of the supporting and positioning portions 112; a plurality of micro flow channels 3, which are arranged in a radiating form at intervals beside the central injection portion 2 so as to allow the specimen to evenly flow into the micro flow channels 3 by means of a centrifugal force during rotation of the test carrier 1; a plurality of reaction receptacles 4, which are respectively arranged in the micro flow channels 3, each of the reaction receptacles 4 having a volume of 20µl-80µl; a plurality of preloaded reagents 41, which are respectively disposed in the reaction receptacles 4, each of the preloaded reagents 41 being an antibiotic susceptibility testing (AST) reagent; a plurality of overflow channels 5, which are respectively arranged at one side of the reaction receptacles 4 that is opposite to the micro flow channels 3 and in communication therewith; a plurality of overflow troughs 6, which are respectively arranged at one side of the overflow channels 5 and in communication therewith; a plurality of narrowing portions 51, which are respectively arranged in the overflow channels 5 and have a passage orifice in communication with the reaction receptacles 4 larger than a passage orifice thereof in communication with the overflow troughs 6; and a plurality of vent holes 7, which are respectively arranged at one side of the overflow troughs 6 and in communication therewith.

A method of use of the antibiotic susceptibility testing reagent disc according to the present invention comprises the following steps:

(a) preloading reagents in test carrier: before a test carrier 1 is packaged, preloaded reagents 41 that are freeze-dried are preloaded into a plurality of reaction receptacles 4 thereof;

(b) introducing specimen: at least two supporting and positioning portions 112 are used to limit a depth that an injection tool is inserted into a central injection portion 2, in order to enable introduction of a specimen into the test carrier 1 through the central injection portion 2;

(c) activating testing device: the test carrier 1 is set in a testing device 8, and the testing device 8 is activated;

(d) splitting specimen: the specimen is caused to flow into each of the reaction receptacles 4 due to action of a centrifugal force and a plurality of micro flow channels 3 arranged in a radiating form at interval at one side of the central injection portion 2;

(e) separating excessive specimen: when the specimen completely flows out of the central injection portion 2, a portion of an individual amount of specimen in each of the reaction receptacles 4 that is larger than a volume thereof flows through an overflow channel 5 into an overflow trough 6, wherein the overflow channel 5 is provided with a narrowing portion 51 to reduce a maximum flow rate flowing into the overflow trough 6;

(f) specimen reacting with preloaded reagents: the specimen in each of the reaction receptacles 4 reacts with the preloaded reagent 41 to cause a color change; and

(g) exhausting gas: gases generated during the process of reaction are exhausted and discharged out of the test carrier 1 through vent holes 7 that are respectively in communication with the overflow troughs 6.

The test carrier 1 is a transparent circular disc, and the material is not limited and can be for example a transparent circular disc of polycarbonate (PC), and comprises, in an illustrative example, an upper disc plate 12 and the lower disc plate 11 that are combined together. A number of supporting and positioning portions 112 are arranged between the upper disc plate 12 and the lower disc plate 11 to fulfill supporting therebetween, and in the instant embodiment, as an example of illustration, the number is two. The lower disc plate 11 is provided, at the center of a bottom thereof, with a positioning portion 111 for pivotally connecting a testing device 8. As an example of illustration, the positioning portion 111 is made in the form of a recess having a downward-facing opening. A central injection portion 2 is arranged in a middle area between the upper disc plate 12 and the lower disc plate 11; and the central injection portion 2 comprises a temporary specimen storage area 21 between the upper disc plate 12 and the lower disc plate 11 and an injection opening 22 formed in a center of the upper disc plate 12 and in communication with the temporary specimen storage area 21. The injection opening 22 is formed between the supporting and positioning portions 112 in order to limit an insertion depth of an injection tool. In the instant embodiment, a pipette is taken as an example for illustrating the injection tool. An arrangement of 36 equally spaced ones is taken as an example for illustrating the micro flow channels 3, and consequently, the number of the reaction receptacles 4, the overflow channels 5, and the overflow troughs 6 is all 36, and in the instant embodiment, they being integrally formed with and extending upwards from an upper surface of the lower disc plate 11 is taken as an example for illustration. An illustrative example of the vent holes 7 is 36 passages respectively connecting between the overflow troughs 6 and a surface of the upper disc plate 12. The preloaded reagents 41 can be a combination of a color indicator, an excipient, a lyoprotectant, and an antibiotic, and in the instant embodiment, an example of the color indicator is resazurin or 2,3,5-triphenyltetrazolium chloride, which are specifically used in AST. The excipient functions to shape and concentrate the preloaded reagent 41 for holding in the reaction receptacle 4, in order to prevent the powder structure from causing the preloaded reagent 41 to spread into other locations or to attach to a wall surface to thereby result in nonuniform concentration among the reaction receptacles 4. A lyoprotectant is used in the freeze-drying process of the preloaded reagents 41 to keep the preloaded reagents 41 dry at low temperature without freezing. The antibiotic is the target of the AST experiment. The narrowing portions 51 are set in a stepped or sloped configuration, and the stepped configuration is taken as an example of illustration in the instant embodiment is a ladder shape. However, the corresponding types of the above components are only examples of the preferred embodiments, and all types with the same function belong to the scope of the present invention and are not limited to the above examples.

Through the above description, the structure of the technology of the present invention can be understood. Based on the corresponding combination of such a structure, pre-test steps can be simplified, and multiple antibiotic susceptibility tests can be performed simultaneously, and the specimens can be evenly distributed with an appropriate amount, and a space for the specimen to independently react and a vent hole 7 are provided, thereby achieving the advantages of simple operation, uniform concentration, sufficient bacterial volume, and avoiding cross-contamination of the specimens and reducing the inability to interpret results due to human operation techniques. It is clearly seen from the drawings that the reaction receptacles 4 are preloaded with the preloaded reagents 41, and as the reagents are pre-introduced before the test carrier 1 has been package, the dosage and concentration do not need to be checked by users, and the preloaded reagents 41 are ones that have been subjected to freeze-drying processing, consistency of dosage and concentration is even higher, and the stability of preservation of the antibiotic is also high, this being conducive to extending the storage time and making it easier to transport, and as such, it is more convenient to carry out AST experiments. For users, the operation steps only include introducing the specimen from the central injection portion 2 and setting the test carrier 1 in a test device 8 to prepare for startup, and thus, the pre-steps are very simple and basically eliminate the problem of human operation. And, as the operation of introducing the specimen is commonly carried out by means of a pipette, while in the present invention, the test carrier 1 is provided with an annular arrangement of the supporting and positioning portions 112, which surround and define a space that serves as the injection opening 22, during the operation, the user only needs to hold the pipette against the injection opening 22 to use the supporting and positioning portions 112 to reduce the opening diameter of the central injection portion 2, and as such, the pipette can be held on the supporting and positioning portions 112 and does not get into contact with a sealing membrane on the bottom. By limiting the insertion depth of the injection tool, it is possible to eliminate the concern about the bottom sealing membrane detaching or breaking resulting from excessively deep insertion and also to ensure that the specimen can be completely injected into the test carrier 1, thereby reducing side leakage and improving the accuracy of the specimen amount.

When the test carrier 1 rotates, the specimen flows into the reaction receptacles 4 through the plurality of micro flow channels 3 that are arranged in a radiating form at intervals due to the centrifugal force. This design allows each of the micro flow channels 3 form an independent channel and the specimen flowing into any one of the micro flow channels 3 does not contact with the specimen in other ones of the micro flow channels 3. Because the specimen is not distributed through natural diffusion and instead, the specimen is driven by the centrifugal force to flow into the reaction receptacles 4, the specimen introduced into the central injection portion 2 can be evenly distributed to each of the micro flow channels 3, making the dosage balanced.

Next, the specimen flows through each of the micro flow channels 3 to the reaction receptacle 4 in the same path. Since the volume of the reaction receptacles 4 is 20μl-80μl, it is relatively sufficient compared to the bacterial amount in the prior art. If the bacterial amount is too small, it is easy to be strongly affected by antibiotics, resulting in the result being unreadable. If the bacterial amount is too large, the antibiotics will be easily outcompeted by the bacteria, resulting in the test results being worthless. Therefore, in the present invention, the reaction receptacles 4 are designed with a volume of 20µl-80µl to provide the most appropriate bacterial amount, which is conducive to performance of a complete and accurate AST experiment, and the instant embodiment takes the optimal volume of 50μl of the reaction receptacles 4 as an example. Of course, when the specimen completely flows out of the central injection portion 2, a portion of the amount of specimen in each of the reaction receptacles 4 that is larger than the volume thereof flows through the overflow channel 5 into the overflow trough 6 to realize easy separation. Therefore, the present invention can allow the condition that a small amount of bacterial liquid overflows the reaction receptacle 4, and the amount of the specimen in each of the reaction receptacles 4 can be stably controlled, in combination with the freeze-dried preloaded reagent 41, making all conditions easy to master and improve the integrity and accuracy of the experiment.

In addition, in the instant embodiment, the extending directions of the micro flow channels 3 and the overflow channel 5 are all toward one side of the central injection portion 2, and for the overflow channel 5 and the micro flow channel 3 on the same reaction receptacle 4, the overflow channel 5 is located on the side of the micro flow channel 3 that is opposite to the rotation direction of the test carrier 1, that is due to the centrifugal force, the bacterial liquid in the reaction receptacle 4 will be concentrated on the side of the reaction receptacle 4 that is away from the central injection portion 2, and the opening of the overflow channel 5 connected to the reaction receptacle 4 is arranged on the side of the reaction receptacle 4 that is close to the central injection portion 2, and therefore, under normal circumstances, the bacterial liquid will not enter the overflow channel 5, and if the rotation direction of the test carrier 1 is clockwise, the overflow channel 5 will be arranged on the counterclockwise side of the micro flow channel 3. In this way, even if there is bacterial liquid in the overflow trough 6, it is difficult to pass through the overflow channel 5 again during the rotation process to get back to the reaction receptacle 4, thereby avoiding a situation of backward flowing. In particular, the present invention provides a narrowing portion 51 on the overflow channel 5, and the passage orifice of the narrowing portion 51 that is connected to the reaction receptacle 4 is larger than the passage orifice thereof connected to the overflow trough 6, that is, the maximum flow rate flowing into the overflow groove 6 is reduced by utilizing the gradually shrinking size of the entry openings, or it can be further arranged to have a path width of the overflow channel 5 narrower than a path width of the micro flow channel 3 to slow down the flow rate into the overflow channel 5, so that the bacterial liquid can be fully mixed with the preloaded reagent 41 before flowing into the overflow trough 6, avoiding a large amount of bacterial liquid flowing forward, and also ensuring the bacterial liquid can be fully mixed with the preloaded reagent 41, and the bacterial liquid flowing into the overflow trough 6 is more difficult to flow back into the micro flow channel 3 again, effectively avoiding the problem of cross contamination.

Subsequently, the specimen reacts with the preloaded reagent 41 in each of the reaction receptacles 4, and the gas generated during the reaction is discharged out of the test carrier 1 through the vent hole 7, thereby preventing the test carrier 1 from compression and collapse caused by an excessive amount of gas happening in a disc of a closed form, leading to damage of the test carrier 1 or the bacterial liquid overflowing to other ones of the micro flow channels 3, and indirectly preventing the bacterial liquid from being contaminated. Through the above operation, the AST experiment can be simply implemented. After that, a result obtained through the color displayed on the indicator of the test carrier 1, judgement of the effectiveness of the antibiotic can be fulfilled.

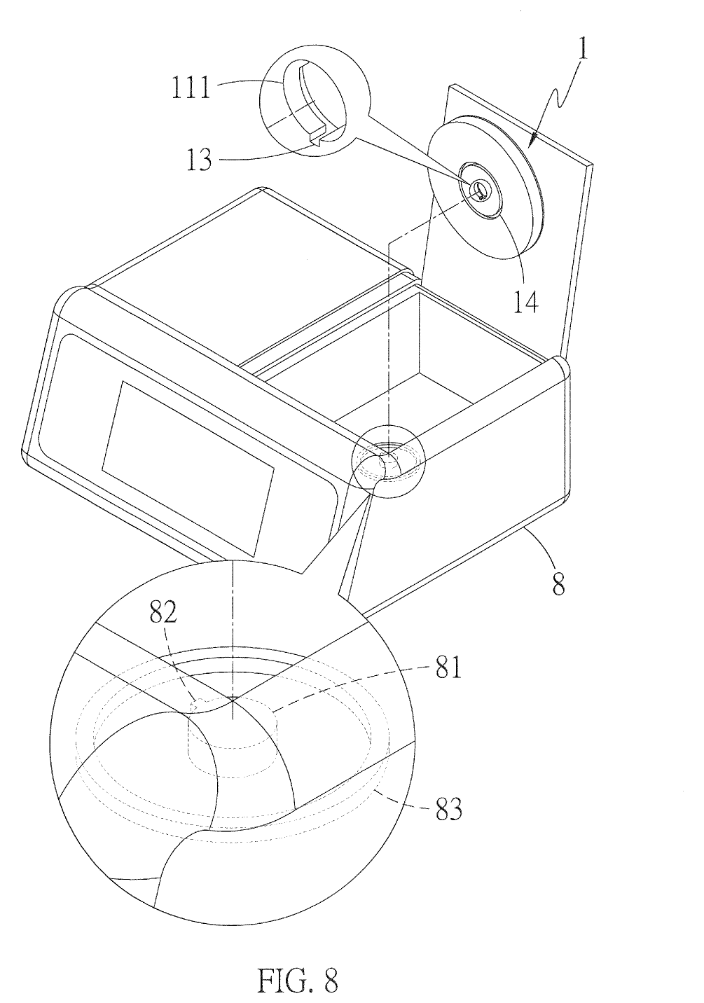

Referring to FIG. 8, which is an exploded view showing a second preferred embodiment of the present invention, it is clearly seen from the drawing that the instant embodiment is similar to the previous embodiment and is only different in that the test carrier 1 is provided with at least one fixing portion 13 for fixing with a testing device 8, and the test carrier 1 is provided with a foolproof portion 14 for identification of a setting direction of the test carrier 1, wherein the fixing portion 13 functions to prevent the test carrier 1 from separating from the testing device 8 during rotation thereof, and the foolproof portion 14 is used to confirm the setting direction of the test carrier 1 to prevent set reversely to result in spilling of the specimen. In the instant embodiment, a constraint track, such as an L-shaped track, arranged on a sidewall of the positioning portion 111 is provided as an example of the fixing portion 13, and the testing device 8 is correspondingly provided with a fixing member 82 in the form of a block on an axle 81 thereof for combination with the fixing portion 13, and in this way, detachment and separation of the test carrier 1 can be effectively prevented. An annular groove formed in the underside of the test carrier 1 is provided as an example of the foolproof portion 14, and thus, the testing device 8 is provided with a foolproof indication member 83 in the form of an annular rib corresponding to the foolproof portion 14. If the user mistakenly sets the test carrier 1 in a reversed way, the foolproof portion 14 is set facing upward, making it not possible for the foolproof indication member 83 to insert into the test carrier 1 and thus the test carrier 1 cannot be fixed by which the user is remind of the reversely setting.

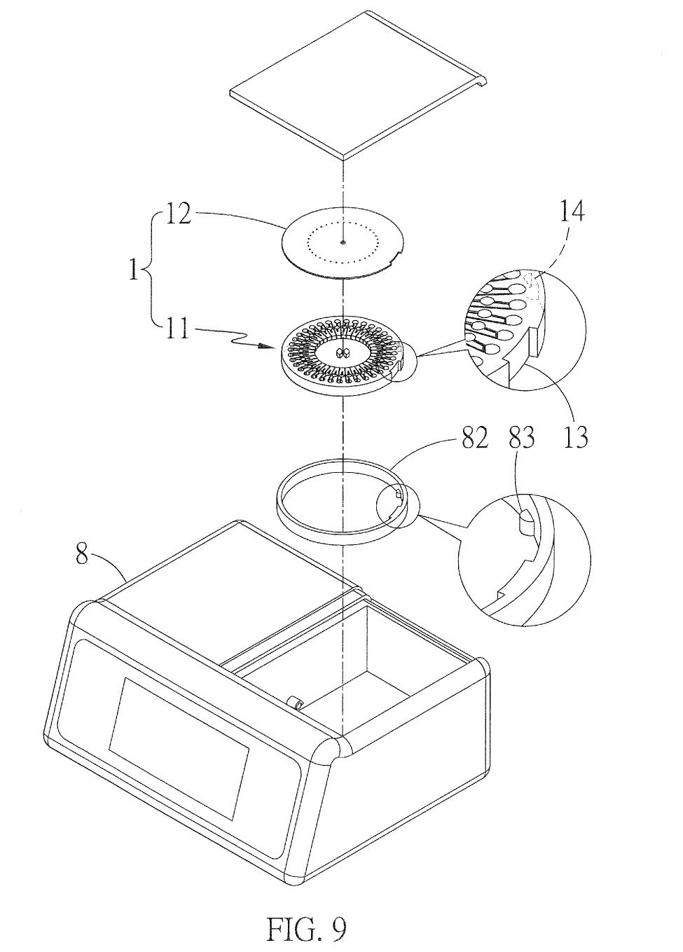

Referring to FIG. 9, which is an exploded view showing a third preferred embodiment of the present invention, it is clearly seen from the drawing that the instant embodiment is similar to the previous embodiments and is only different in that the rotation of the test carrier 1 and the corresponding arrangement between the foolproof portion 14 and the fixing portion 13 are changed. In the instant embodiment, the test carrier 1 does not include, on the underside thereof, a positioning portion serving as an axis, and the testing device 8 does not have an axle pivotally connected to the test carrier 1, yet the testing device 8 is instead provided with a fixing member 82 in the form of a circular ring that is drivable to rotate from an outer circumference thereof by an electrically-driven roller for positioning, and this allows the test carrier 1 to be set inside the fixing member 82 while still drivable through the fixing portion 13 to rotate so as to achieve the purpose of preventing detachment and separation during rotation. The foolproof portion 14 is formed, as an example, as a notch in a side edge of the lower disc plate 11, and the fixing member 82 is provided, on a lower edge of an inside wall, with a foolproof indication member 83 in the form of a block protruding inwards. In this way, as the upper disc plate 12 does not include the foolproof portion 14, when the user mistakenly set the test carrier 1 in a reversed direction, interference may be caused with the foolproof indication member 83, making it not possible to dispose therein, thereby achieving the purpose of foolproofness.

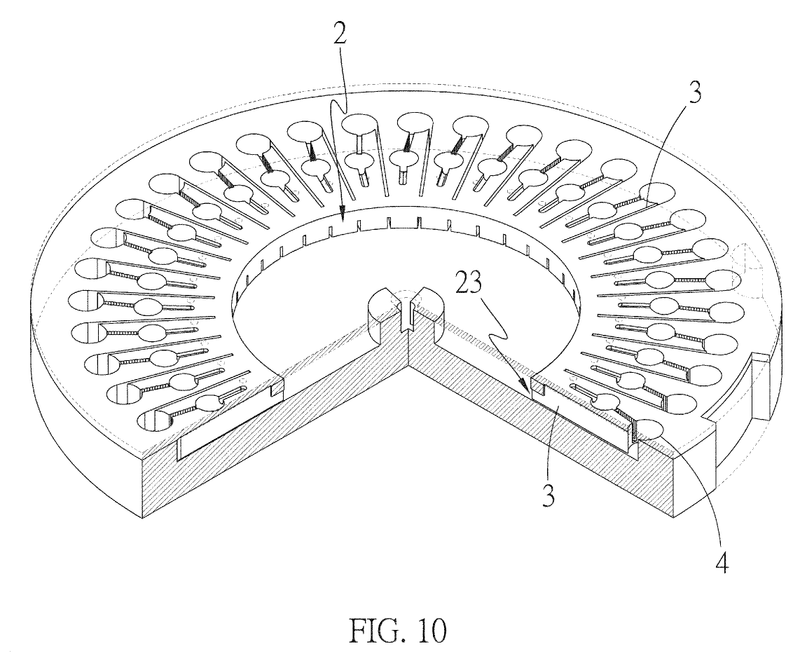

Referring to FIG. 10, which is a perspective view, partly sectioned, showing a fourth preferred embodiment of the present invention, it is clearly seen from the drawing that the instant embodiment is similar to the previous embodiments and is only different in that an outer circumference of the central injection portion 2 is provided, on one side thereof communicating with the micro flow channels 3, with a flow-limiting groove 23. The flow-limiting groove 23 is a flattened annular slit that has a height less than a height of the micro flow channels 3, and is, specifically speaking, a drainage opening formed by recessing from a bottom of the wall of the central injection portion 2 toward interior of the wall, and as such, regardless of the amount of the specimen flowing toward the micro flow channels 3 at an instant, the amount of the specimen entering the micro flow channels 3 is limited by the arrangement of the flow-limiting groove 23, and since the flow-limiting groove 23 is of an annular form that connects an entry opening of each of the micro flow channels 3, when a large amount of specimen rushes to the entry opening of a specific one of the micro flow channels 3, the flow-limiting groove 23 can block it immediately and can also guide the excess specimen toward two sides so as to have the specimen in the central injection portion 2 first concentrated in the flow-limiting groove 23 and then slowly flowing into each of the micro flow channels 3, thereby making the specimen more evenly distributed among individual ones of the reaction receptacles 4, and also reducing the amount of specimen remaining in the central injection portion 2.

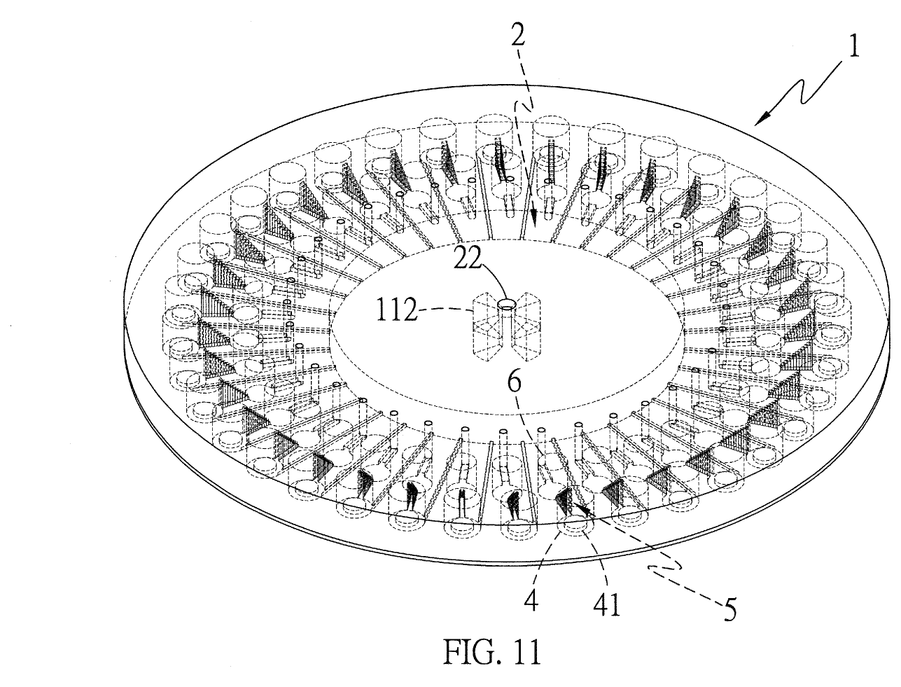

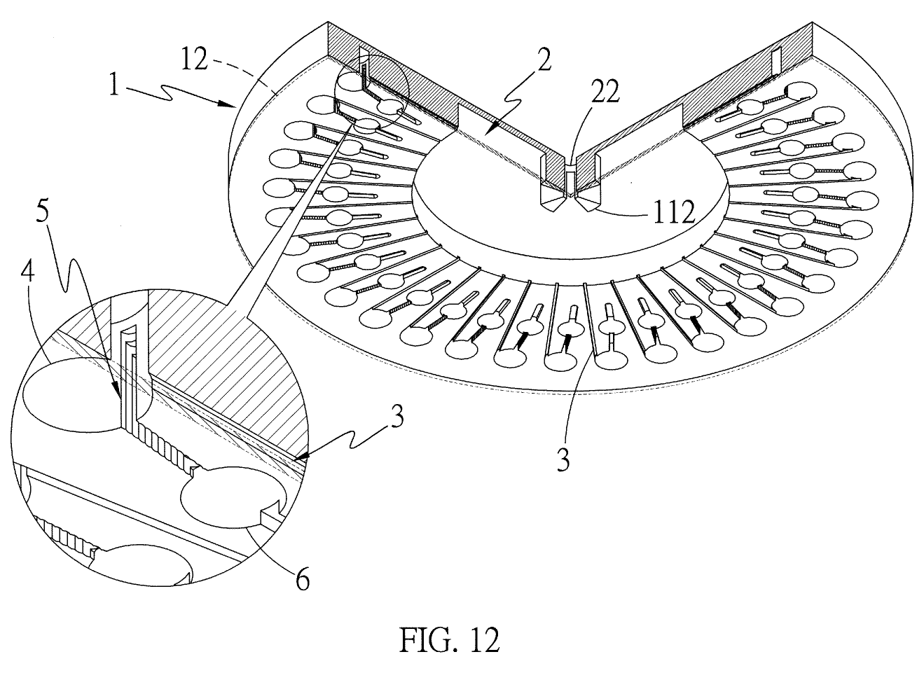

Referring simultaneously to FIGS. 11 and 12, which are respectively a perspective view, in a see-through form, showing a fifth preferred embodiment of the present invention and a perspective view, partly sectioned, showing the fifth preferred embodiment of the present invention from a different angle, it is clearly seen from the drawings that the instant embodiment is similar to the previous embodiments and is only different in that each of the micro flow channels 3 has a cross-sectional area of 100µm-300µm square, and as such, the micro flow channels 3 only form relatively shallow trenches in the surface of the test carrier 1, and to combine with the testing device, the test carrier 1 is reversed to have the micro flow channels 3 and a sealing membrane (the upper disc plate 12) both located on the lower side, while the injection opening 22 of the central injection portion 2 is formed in the bottom of the test carrier 1 and will be exposed on the upper side when reversely set. Further, the number of the supporting and positioning portions 112 is increased to four. As such, the size of the micro flow channels 3 is significantly reduced. As an example of the instant embodiment, the cross-sectional area of the micro flow channels 3 is 200 x 200µm², making the effect of reducing the flow speed of the bacterial liquid flowing into the overflow channels 5, the effect of the bacterial liquid mixing with the preloaded reagents 41, the effect of suppressing a large quantity of bacterial liquid flowing in forwards, the effect of preventing the bacterial liquid from flowing again back to the micro flow channels 3, and the effect of preventing cross-contamination can all be enhanced. As to the communication of the reaction receptacles 4 with the bottom of the overflow troughs 6 resulting from reversely setting, since the previously described arrangement of using the centrifugal force and the overflow channels 5 being located at one side of the micro flow channels 3 that is opposite to the rotation direction of the test carrier 1, basically there is no problem of bacterial liquid surging into the overflow troughs 6.

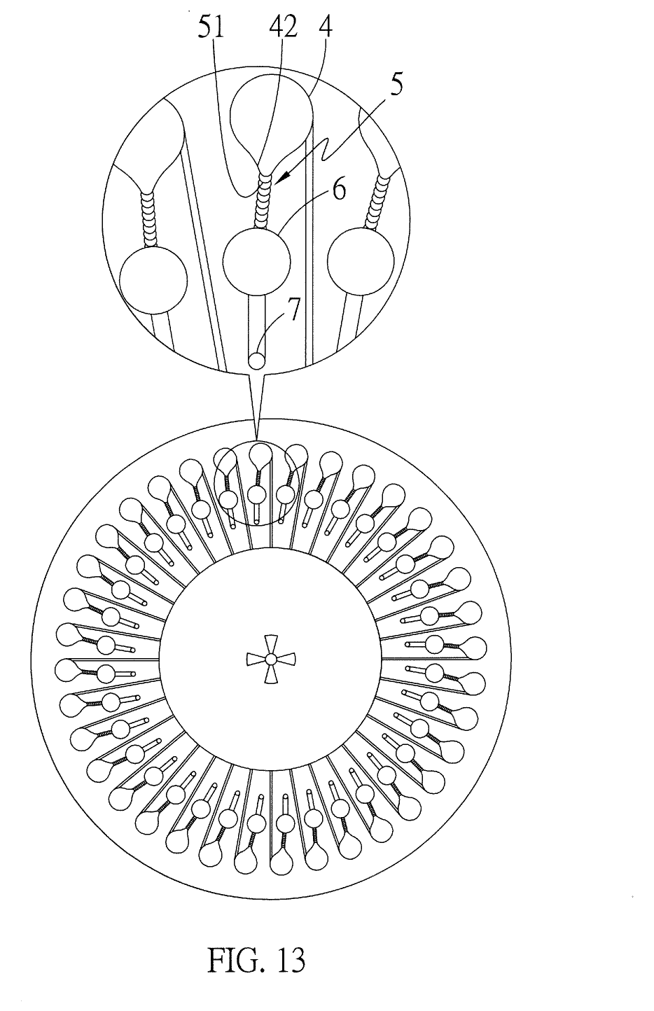

Referring to FIG. 13, which is a plan view showing a sixth preferred embodiment of the present invention, it is clearly seen from the drawing that the instant embodiment is similar to the previous embodiments and is only different in that a convergence groove 42 is arranged at the site where each of the reaction receptacles 4 is connected with the respective one of the overflow channels 5. The convergence groove 42 is of a slightly convex form, making the reaction receptacle 4 into a water droplet form when combined therewith. The narrowing portion 51 is connected to one side of the convergence groove 4. As such, when the amount of the bacterial liquid in the reaction receptacle 4 exceeds the volume thereof, it will be first concentrated at the convergence groove 42 so as to immediately direct excess specimen to flow toward the overflow trough 6, achieving balance between limiting the specimen from unexpectedly flowing into the overflow trough 6 and preventing the specimen from massively flowing into the overflow trough 6. The arrangement of the convergence groove 42 makes it possible to guide excess specimen, if any, to flow into the overflow trough 6, and also to work with the narrowing portion 51 under a normal condition in order to prevent the specimen from incorrectly flowing into the overflow trough 6, also using the concentration effect realized with the convergence groove 42 to push gas, in the form of gas bubbles, from the back side of the overflow trough 6 in a direction toward the vent hole 7 to enhance the effect of gas discharging.

I claim:

1. An antibiotic susceptibility testing (AST) reagent disc structure, comprising:
a test carrier;
at least two supporting and positioning portions, which are arranged in the test carrier to support the test carrier;
a central injection portion, which is arranged among the supporting and positioning portions for introducing a specimen, wherein an insertion depth of an injection tool is limitable by means of the supporting and positioning portions;
a plurality of micro flow channels, which are arranged in a radiating form at intervals beside the central injection portion to be each in communication therewith, so that during rotation of the test carrier, the specimen is caused by a centrifugal force to evenly flow into each of the micro flow channels;
a plurality of reaction receptacles, which are respectively arranged on the micro flow channels, each of the reaction receptacles having a volume of 20µl-80µl;
a plurality of preloaded reagents, which are respectively loaded in the reaction receptacles, each of the preloaded reagents being an AST reagent;
a plurality of overflow channels, which are respectively formed at one side of the reaction receptacles that is opposite to the micro flow channels and are in communication therewith;
a plurality of overflow troughs, which are respectively formed at one side of the overflow channels and are in communication therewith;
a plurality of narrowing portions, which are respectively formed on the overflow channels and having a passage orifice connecting with the reaction receptacles and larger than a passage orifice thereof connecting with the overflow troughs; and
a plurality of vent holes, which are respectively formed at one side of the overflow troughs and are in communication therewith.

2. The AST reagent disc structure according to claim 1, wherein the test carrier comprises at least one fixing portion for fixing to a testing device, and the test carrier comprises a foolproof portion for identifying a setting direction of the test carrier.

3. The AST reagent disc structure according to claim 1, wherein a convergence groove is arranged at one side of each of the reaction receptacles connecting with a respective one of the overflow channels.

4. The AST reagent disc structure according to claim 1, wherein the micro flow channels each have a cross-sectional area of 100µm-300µm square.

5. The AST reagent disc structure according to claim 1, wherein one side of an outer circumference of the central injection portion connecting with the micro flow channels is provided with a flow-limiting groove.

6. A method of use of antibiotic susceptibility testing (AST) reagent disc, of which steps comprise:
(a) before a test carrier is packaged, preloading freeze-dried preloaded reagents in a plurality of reaction receptacles thereof;
(b) limiting a depth of an injection tool inserting into a central injection portion by using at least two supporting and positioning portions, in order to introduce a specimen through the central injection portion into the test carrier;
(c) positioning the test carrier in a testing device and activating the testing device;
(d) the specimen being acted upon by a centrifugal force to flow through a plurality of micro flow channels arranged in a radiating form at interval beside the central injection portion into each of the reaction receptacles;
(e) after the specimen completely flows out of the central injection portion, a portion of an individual amount of specimen in each of the reaction receptacles that is larger than a volume thereof flowing through an overflow channel into an overflow trough, the overflow channel including a narrowing portion to reduce a maximum flow rate flowing into the overflow trough;
(f) the specimen in each of the reaction receptacles reacting with the preloaded reagent to cause a color change; and
(g) gas generated during reaction discharging out of the test carrier through a vent hole in communication with the overflow trough.

7. The method of use of AST reagent disc according to claim 6, wherein in Step (c), in positioning the test carrier in the testing device, a foolproof portion provided on the test carrier is applied to identify a direction of setting, and in Step (d), during rotation of the test carrier, at least one fixing portion provided on the test carrier is applied to prevent detachment and separation of the test carrier.

8. The method of use of AST reagent disc according to claim 6, wherein a convergence groove is arranged at one side of each of the reaction receptacles connecting with a respective one of the overflow channels.

9. The method of use of AST reagent disc according to claim 6, wherein the micro flow channels each have a cross-sectional area of 100µm-300µm square.

10. The method of use of AST reagent disc according to claim 6, wherein one side of an outer circumference of the central injection portion connecting with the micro flow channels is provided with a flow-limiting groove.
